(19) United States
(12) Patent Application Publication
(10) Pub. No.: US 2011/0117605 A1
(43) Pub. Date: May 19, 2011

(54) Methods for Manufacturing a Polyclonal Protein

(75) Inventors: Anne Bondgaard Tolstrup, Hilleroed (DK); Lars Soegaard Nielsen, Nivaa (DK); Dietmar Weilguny, Virum (DK); Christian Müller, Vaerloese (DK); Finn C. Wiberg, Farum (DK); Jonas Heilskov Graversen, Hornslet (DK)

(73) Assignee: Symphogen A/S, Kgs. Lyngby (DK)

(21) Appl. No.: 12/989,340

(22) PCT Filed: Apr. 23, 2009
(86) PCT No.: PCT/DK2009/050094

§ 371 Date: Jan. 4, 2011

Related U.S. Application Data
(60) Provisional application No. 61/047,389, filed on Apr. 23, 2008.

(30) Foreign Application Priority Data
Apr. 23, 2008 (DK) PA 2008 00580

(51) Int. Cl. C12P 21/00

(52) U.S. Cl. 435/71.2; 435/71.1

(57) ABSTRACT

The invention relates to methods for manufacturing drug products comprising at least two distinct members of a polyclonal protein, for example a polyclonal antibody, where each distinct member is expressed by a separate population of cells. The methods involve at least an initial step in which the cell populations expressing the distinct members of the polyclonal protein are cultured separately. The individual cell populations, or proteins expressed by the individual cell populations, are combined at a later point of the upstream or downstream processing to result in a single drug product comprising the distinct members of the polyclonal protein.

### FIELD OF THE INVENTION

The present invention relates to methods for manufacturing drug products comprising at least two distinct members of a polyclonal protein. The invention involves at least an initial separate culturing step of cells expressing the distinct members of the polyclonal protein. The cell lines or protein preparations are combined at a later point upstream or prior to or during downstream processing, ending ultimately with one drug product comprising at least two distinct members of a polyclonal protein.

### BACKGROUND OF THE INVENTION

Manufacturing of recombinant polyclonal proteins is a relatively new technology. The vast majority of recombinant drug products are manufactured as monoclonal products using a selected cell line derived from one clonal cell expressing the desired product. The upstream and downstream manufacturing methods in this case can be optimised for the specific monoclonal drug product.

Cost effective methods for manufacturing recombinant antibodies and other protein products in vivo using transgenic animals expressing the recombinant product in milk (mammals) or eggs (chickens) or transgenic plants expressing the recombinant product throughout the plant or in a separate organ (tuber, seed, leaf) have been developed in recent years. However, these methods have yet to be adapted to the manufacture of polyclonal protein products.

One method for manufacturing a polyclonal drug product is disclosed in WO 2004/009618, which concerns the use of a monoclonal cell line expressing antibodies having identical light chains. According to this document, a mixture of antibodies can be produced by expression in a recombinant host of a nucleic acid sequence encoding a common light chain and nucleic acid sequences encoding at least two different heavy chains with a different variable region capable of pairing with the common light chain.

WO 2004/061104 provides a solution to the problem of scrambling of heavy and light antibody chains which is inherent to the method described in WO 2004/009618, by having only one expression construct integrated at a specific location of the genome of the expression cells using site-specific integration. This solution enables the generation of a polyclonal manufacturing cell line which is able to express distinct members of a polyclonal protein, so that the polyclonal protein, e.g. a polyclonal antibody, can be manufactured in a single batch using one cell bank, which is expanded in one bioreactor. The supernatant is subsequently purified using one downstream purification method, resulting in one drug substance that is formulated into one drug product. Examples of recombinant polyclonal antibodies manufactured using this method include a recombinant polyclonal anti-RhesusD antibody (WO 2006/007850) and a recombinant polyclonal anti-Vaccinia virus antibody (WO 2007/065433).

WO 2008/145133 describes a method for manufacturing a recombinant polyclonal protein composition, in particular a recombinant polyclonal antibody composition, by means of random integration, wherein host cells are separately transfected with a set of expression vectors each comprising at least one copy of a distinct nucleic acid encoding a distinct member of the polyclonal protein under conditions that avoid site-specific integration of the expression vectors into the genome of the cells.

Despite these advances in manufacturing of polyclonal proteins, there remains a need for providing alternative manufacturing methods, e.g. for cases where one or more members of the polyclonal protein requires particular conditions for expression and/or purification, or for conditions where separate upstream processing is preferred.

### SUMMARY OF THE INVENTION

In a first aspect the invention relates to a method for manufacturing a drug product comprising at least two distinct protein members of a polyclonal protein, said method comprising the steps of:
a) providing at least two populations of cells, wherein each population encodes one distinct member of the polyclonal protein and is enclosed in a physically separate container comprising culture medium and cells expressing the protein, wherein the method comprises an upstream part comprising the steps of:
   i) expanding the at least two populations of cells in one or more steps of a seed train in separate containers;
   ii) expanding cells from the seed train in one or more steps of an inoculum train;
   iii) culturing cells from the inoculum train in a production phase under conditions favoring expression of the protein members so as to express the at least two distinct protein members;
wherein the at least two populations of cells are kept separate at least during the seed train;
b) harvesting the expressed protein;
c) performing at least one purification step on the harvested protein;
d) obtaining purified drug substance; and
e) formulating purified drug substance into a polyclonal drug product.

The characterising feature of the method is that the at least two populations of cells each encoding one distinct member of the polyclonal protein are kept separate in physically separate containers at least during the initial phases of cell expansion. In other embodiments as will be described herein, the populations of cells expressing the distinct members of the polyclonal protein are kept separate during a longer part or during the entire part of the upstream processing. In still further embodiments, the expressed distinct members of the polyclonal protein are kept separate during part of or possibly the whole downstream processing, which is completed when purified drug substance is obtained. Advantages of the different embodiments are described in detail below.

The at least two different populations of cells may be kept separate at least up to and including the inoculum train, and in further embodiments the at least two different populations of cells are kept separate at least up to and including the production phase.

The at least two distinct protein members expressed by the at least two populations of cells may further be kept separate at least up to and including the protein harvest, so that each distinct member can be harvested separately.

In some embodiments, the at least two distinct protein members expressed by the at least two populations of cells are kept separate at least up to and including at least one purification step, allowing for different initial purification steps of different distinct members. For example, the protein members may be kept separate at least up to and including an affinity chromatography step.

The invention also includes embodiments wherein the at least two distinct protein members expressed by the at least two populations of cells are kept separate at least up to and including obtaining the drug substance.

As an alternative to production of the polyclonal protein in single-celled organisms in separate containers, production may take place in physically separate units in the form of multicellular organisms, each multicellular organism expressing one member of the polyclonal protein. These multicellular organisms may be plants, in which case the physically separate unit is a plant or a plant organ. The multicellular organisms may also be a transgenic non-human animal, such as a bird that has been genetically modified to express and secrete a distinct member of the polyclonal protein to an egg. The transgenic non-human animal may alternatively be a mammal, where the distinct member of the polyclonal protein is secreted to the milk. The mammal may for example be a sheep, goat, cow, camel or buffalo.

### DEFINITIONS

Protein production in mammalian cells, e.g. CHO (Chinese hamster ovary) cells, may employ a semi-continuous process whereby cells are cultured in a “seed train” for various periods of time and are subsequently transferred to inoculum fermentors (“inoculum train”) to initiate the cell amplification process en route to larger scale production of the protein of interest. Thus, cells used for protein production are in culture for various periods of time up to a maximum predefined cell age. The parameters of the cell culture process, such as seed density, pH, DO₂ and temperature, duration of the production culture, operating conditions of harvest, etc., are a function of the particular cell line and culture medium used, and can be determined empirically without undue experimentation. For the purposes of the present invention the “seed train” phase includes the steps from thawing of the cells and the initial expansion steps. These steps are typically carried out in Petri dishes, shaker flasks, T-flasks, plastic bags, spinner flasks, centrifuge tubes, multiwell plates, roller bottles, or other bottles, i.e. containers that are not equipped with tubing to transfer samples from one container to the other. The “inoculum train” starts when the first bioreactor or fermentor (steel bioreactor or disposable bioreactor) is inoculated with cells from the “seed train”. Once cells have been transferred to the inoculum bioreactor, subsequent transfer to other bioreactors typically takes place through a closed tubing system that allows cells to be pumped from one container to the other.

“Drug product” means a finished dosage form, for example, tablet, capsule, solution or lyophilized product, that contains a “drug substance”, generally, but not necessarily, in association with one or more other ingredients. A “drug substance” is usually manufactured in large batches, which are subsequently formulated into a “drug product” by combining it with other ingredients. In the context of the present invention, the drug product will contain the final polyclonal protein mixture comprising the distinct members of the polyclonal protein. Depending on the particular manufacturing scenario, the drug substance may also be a polyclonal mixture containing all of the distinct members of the polyclonal protein, or a drug substance may contain one or only some of the distinct members of the polyclonal protein. In the latter case, two or more drug substances will be formulated into a single drug product.

The term “container” or “vessel” refers to a vessel or container adapted for in vitro culturing of cells. Examples of containers include Petri dishes, shaker flasks, T-flasks, plastic bags, spinner flasks, centrifuge tubes, multiwell plates, roller bottles, other bottles, bioreactors of steel or other non-disposable bioreactors, and plastic or other disposable bioreactors.

By “protein” or “polypeptide” is meant any chain of amino acids, regardless of length or post-translational modification. Proteins can exist as monomers or multimers, comprising two or more assembled polypeptide chains, fragments of proteins, polypeptides, oligopeptides or peptides.

As used herein, the term “polyclonal protein” or “polyclonality” refers to a protein composition comprising different, but homologous protein molecules, preferably selected from the immunoglobulin superfamily. Thus, each protein molecule is homologous to the other molecules of the composition, but also contains one or more stretches of variable polypeptide sequence which are characterized by differences in the amino acid sequence between the individual members of the polyclonal protein. Known examples of such polyclonal proteins include antibody or immunoglobulin molecules, T cell receptors and B cell receptors. A polyclonal protein may consist of a defined subset of protein molecules defined by a common feature such as shared binding activity towards a desired target, e.g., in the case of a polyclonal antibody against the desired target antigen.

The term “a distinct member of a recombinant polyclonal protein” denotes one protein molecule of a protein composition comprising different, but homologous protein molecules, where each protein molecule is homologous to the other molecules of the composition, but also contains one or more stretches of variable polypeptide sequence which are characterized by differences in the amino acid sequence between the individual members of the polyclonal protein.

Each “population of cells” that encodes one distinct member of the polyclonal protein is derived from an individual monoclonal cell bank or monoclonal cell culture, where the monoclonal cell bank or monoclonal cell culture is derived from a single cell as described elsewhere herein.

The term “harvest” as used herein refers to harvest of the supernatant containing the expressed protein, whereas subsequent steps to isolate the desired protein from the supernatant, including clarification, are generally considered to be purification steps.

The term “antibody” describes a functional component of serum and is often referred to either as a collection of molecules (antibodies or immunoglobulin) or as one molecule (the antibody molecule or immunoglobulin molecule). An antibody molecule is capable of binding to or reacting with a specific antigenic determinant (the antigen or the antigenic epitope), which in turn may lead to induction of immunological effector mechanisms. An individual antibody molecule is usually regarded as monospecific, and a composition of antibody molecules may be monoclonal (i.e., consisting of identical antibody molecules) or polyclonal (i.e., consisting of different antibody molecules reacting with the same or different epitopes on the same antigen or even on distinct, different antigens). Each antibody molecule has a unique structure that enables it to bind specifically to its corresponding antigen, and all natural antibody molecules have the same overall basic structure of two identical light chains and two identical heavy chains. Antibodies are also known collectively as immunoglobulins. The terms antibody or antibodies as used herein are also intended to include chimeric and single chain antibodies, as well as binding fragments of antibodies, such as Fab, Fv fragments or scFv fragments, and multimeric forms such as dimeric IgA molecules or pentavalent IgM.

The term “immunoglobulin” is commonly used as a collective designation of the mixture of antibodies found in blood or serum, but may also be used to designate a mixture of antibodies derived from other sources.

The term “polyclonal antibody” describes a composition of different antibody molecules which are capable of binding to or reacting with several different specific antigenic determinants on the same antigen or on different antigens. Usually, the variability of a polyclonal antibody is thought to be located in the so-called variable regions of the polyclonal antibody. However, in the context of the present invention, polyclonality can also be understood to describe differences between the individual antibody molecules residing in so-called constant regions, e.g. as in the case of mixtures of antibodies containing two or more antibody isotypes such as the human isotypes IgG1, IgG2, IgG3, IgG4, IgA1 and IgA2, or the murine isotypes IgG1, IgG2a, IgG2b, IgG3 and IgA. The term “polyclonal antibody” as used herein can also be thought of as a mixture of two or more monoclonal antibodies.

The term “a library of variant nucleic acid molecules of interest” is used to describe a collection of nucleic acid molecules which collectively encode a “recombinant polyclonal protein of interest”. When used for transfection, the library of variant nucleic acid molecules of interest is contained in a library of expression vectors. Such a library typically has at least 3, 5, 10, 20, 50, 1000, 10⁴, 10⁵ or 10⁶ distinct members.

As used herein, the term “operably linked” refers to a segment being linked to another segment when placed into a functional relationship with the other segment. For example, DNA encoding a signal sequence is operably linked to DNA encoding a polypeptide if it is expressed as a leader that participates in the transfer of the polypeptide to the endoplasmic reticulum. Also, a promoter or enhancer is operably linked to a coding sequence if it stimulates the transcription of the sequence.

The term “transfection” is herein used as a broad term for introducing foreign DNA into a cell. The term is also meant to cover other functional equivalent methods for introducing foreign DNA into a cell, such as transformation, infection, transduction, or fusion of a donor cell and an acceptor cell.

The terms “variable polypeptide sequence” and “variable region” are used interchangeably.

The term “head-to-head promoters” refers to a promoter pair being placed in close proximity so that transcription of two gene fragments driven by the promoters occurs in opposite directions. A head-to-head promoter can also be constructed with a stuffer composed of irrelevant nucleic acids between the two promoters. Such a stuffer fragment can easily contain more than 500 nucleotides. Head-to-head promoters can also be termed bi-directional promoters.

The term “plant” refers to an organism that is a member of the Plantae kingdom. A plant for the purposes of the present invention includes those organs of a plant that are required for the independent growth of a plant (e.g. roots, stem, leaves). When the term plant is intended to cover isolated plant cells, this is clearly stated.

### DESCRIPTION OF THE DRAWINGS

FIG. 1: Schematic illustration of a production scenario of the invention (separate seed/inoculum train).

FIG. 2: Schematic illustration of another production scenario of the invention (separate production phase).

FIG. 3: Relative area of each of six anti-vaccinia antibodies after 7 days of cultivation in a mixed population (for details see Example 1).

FIG. 4: Relative amounts of six different anti-RSV antibodies after 12 days of cultivation in a mixed population (see Example 3).

### DETAILED DESCRIPTION OF THE INVENTION

Manufacturing of recombinant polyclonal antibodies expressed in producer cells generated by integration of the expression construct can be performed as a single batch production. Advantageously, the individual cell lines or cell clones are properly selected for similar growth rates prior to mixing of the cell lines in one culture container in order to avoid outgrowth of one or a few of the cell lines.

There may be situations where it proves difficult or impossible to align the growth rates of individual cell lines in the desired manner, for example if production of a particularly desired recombinant protein/antibody influences the growth rate of the cell line in a negative direction. Also, there may be situations where very distinct ratios of the different members of the polyclonal protein are desirable. Such cases could e.g. be where very unequal amounts of the polyclonal protein members in the composition are desirable, for example due to different affinities to the target. For example, if a polyclonal composition containing six protein members with ratios between the individual members of e.g. 20:5:1:1:1:0.5 is desired, it would be more feasible to control and obtain such a composition by keeping the distinct members separate during at least part of the manufacturing process. Another example could be in cases where two or more different parental cell lines had been used for transfection and expression of the distinct recombinant products, in which case the cell culture conditions for the two or more cell lines could be different, for example the optimal medium composition for expansion of the cells and/or expression of the distinct member of the polyclonal protein.

An alternative method for manufacturing of such recombinant proteins which can help circumvent the requirement for similar growth rates of individual expression cell lines is to keep the cell lines separate at least during the seed train part of the upstream process. The cell lines expressing different antibodies may then be combined at a later stage, e.g. prior to or during the inoculum train, or prior to or during the production phase. One advantage of this method is that the number of population doublings of the mixed cell culture can be dramatically reduced, which makes it easier to control the ratios of the individual members of the polyclonal protein in the final drug product, even if the individual cell lines exhibit different growth rates. The separate manufacturing may be continued during one or more steps of the downstream processing, and the distinct protein members may even be kept separate up to the point where the drug substance has been purified to the extent required. In the latter case, the drug substances are then combined to form the final drug product, which comprises at least two distinct members of a polyclonal protein, such as two or more different antibodies targeting the same or different antigens.

One important element of the invention is that whether combination takes place during growth of the cell lines, or at a point where proteins have been separated from the cell lines, the final product is always a single polyclonal drug product comprising at least two distinct members of a polyclonal protein, such as two or more different antibodies targeting the same or different antigens.

In one embodiment the individual members of the polyclonal protein are kept separate until harvest of the supernatant containing the individual recombinant proteins. These products are then mixed prior to downstream purification to simplify the CMC process and reduce costs.

In cases where the individual members of the polyclonal protein cannot be purified using the same methods, they can also be mixed at a later stage during the downstream purification process or even upon completion of purification prior to preparing the final drug product.

It will be apparent that the individual members of the polyclonal protein, if not mixed at an earlier stage or manufactured together after mixing of two or more populations of cells, will be mixed together no later than during formulation of the polyclonal drug product.

Manufacturing of the individual expression cell lines producing each of the components of the final product is preferentially done by cultivation in low-cost equipment such as disposable bioreactors, e.g. Wave Bags, which are commercially available in a number of different bag sizes, but it is also possible to run multiple non-disposable bioreactors in cases where the volume of the individual production runs are low, for example below 50 L or preferentially below 20 L or 10 L. The cultivation of recombinant protein from each of the individual cell lines is preferentially done in parallel, but may also be performed sequentially in two or more groups.

The inoculation material for each manufacturing container (e.g. Wave Bags or other types of bioreactors) is obtained by first preparing a Master Cell Bank (MCB), potentially followed by a Working Cell Bank (WCB), for each individual protein producer cell line or clone. A vial from each of the WCBs (or MCBs) is used for seeding of separate culture containers. The seed train is designed for expansion of each of these cell lines until enough cells for inoculation of the inoculum bioreactor has been obtained.

When feasible, the separate part of the manufacturing is preferentially performed using the same conditions for expression of different protein members, for example including the same medium ingredients and added feed solutions (such as glucose, glutamine, potential selective agents such as G418, vitamins, minerals, proteins, hydrolysates, etc.), as well as cultivation process parameters such as pH, DOT (dissolved oxygen tension), CO₂ pressure and temperature. This is important in order to facilitate the quality control process, where factors such as host cell protein reduction, removal of added selective agents and removal of added growth factors need to be addressed. It is also highly important in order to obtain similar post-translational modifications of the recombinant proteins, such as the glycosylation pattern.

Similarly, any separate part of the purification is preferably performed using similar columns, buffers, elution profiles, etc. to the extent possible.

To enable a smooth, cost- and time-efficient downstream and analytical process, it is preferable that the physico-chemical characteristics of the individual distinct protein members in the polyclonal composition are as similar as possible. This is best ensured by keeping the constant parts of the molecules identical. In the case of antibodies, the same expression vector is preferentially used to produce each of the individual recombinant antibody molecules, and the primary sequences of these differ preferentially only in the variable region of the heavy and the light chain, respectively. In the most preferred scenario, the constant parts of the antibody molecules, as encoded by the expression construct, are exactly the same for all members of the polyclonal antibody composition, for example an IgG1 isotype molecule. In addition, the light chains are also preferably of the same type, either Kappa or Lambda in the case of human antibodies. Keeping the major part of the antibody molecules as identical as possible enables purification of the recombinant antibodies to high purity using one procedure without the risk of losing diversity. It also facilitates the establishment of analytical methods needed for characterization, quality control and release of manufactured drug product.

The protein purification process is typically a multi-step process which is developed based on physico-chemical characteristics of the antibody or other protein molecules, and for each step there is a risk of loss of diversity, i.e. of losing one or more of the individual product components. The process may e.g. include several chromatographic steps, including a capture step which could be protein A or, alternatively, protein G binding, an anion exchange chromatography step and a chromatography step based on hydrophobic interaction. It may further include incubation at low pH as well as several filtration steps. It is evident for those skilled in the art that differences in any of these physico-chemical characteristics between the molecules may prohibit the successful recovery of a pure and diverse polyclonal antibody product.

Concerning analytical quality control methods it is evident to those skilled in the art that all the characterization assays necessary for gaining regulatory approval of a recombinant antibody product, for example peptide mapping, size exclusion, SDS-PAGE, Western Blot, amino acid analysis, mapping of disulphide bridges and free sulphydryl groups and mono- and oligo-saccharide characterization, may prove difficult to perform to a satisfactory degree if the constant region is not identical or similar among the molecules. Methods for structural characterization of recombinant polyclonal antibodies and other polyclonal proteins, and for characterization of polyclonal cell lines expressing such polyclonal proteins, are disclosed in WO 2006/007853.

Different Manufacturing Scenarios

Recombinant Expression in Culture Containers

Cultivated mammalian cells have become the dominant system for the manufacturing of recombinant proteins for clinical applications because of their capacity for proper folding, assembly and post-translational modification. Two main formats have been employed for the production of recombinant proteins in mammalian cells: Cultures of adherent cells and suspension cultures. The latter is by far the most common.

In a simple batch or fed batch production process the scale-up to very large volumes can occur by the dilution of the content of a bioreactor into 5-20 volumes of fresh medium held prewarmed in a larger reactor. The process from thawing of banked cells to the actual large-scale production consists of three separate phases—seed train, inoculum train and production phase. The seed train is usually performed in smaller cell culture vessels starting from small volumes of a few mL of culture right after thawing of the banked cells and up to several liters of cell culture, such as 5-100 L of culture, to provide fresh cells for scale-up during the period chosen for the production. The inoculum train starts when the cell suspension generated during the seed train is transferred to the inoculum reactor (also termed seed reactor) and its volume is expanded so that a sufficient cell number is generated for inoculation of the final production bioreactor (Wurm, 2004, Nature Biotechnology, 22 (11):1393-1398).

For manufacturing of monoclonal recombinant proteins it is typically possible to maintain the thawed cells in the seed train stage or alternatively in the inoculum reactor for an extended number of population doublings. This provides the possibility of seeding more than one production bioreactor with cells thawed from the same ampoule, and it also provides backup cells for situations where the manufacturing is subject to delays, for example due to technical issues. However, when manufacturing recombinant polyclonal proteins it is necessary to set strict limits on and maintain thorough control over the number of generations, and it is typically not possible to maintain a seed train from a polyclonal cell bank for extended time periods. This is due to the fact that even when growth rates of different cell lines are matched as closely as possible, the individual cell lines in the polyclonal cell composition will always exhibit slightly different growth and productivity rates, leading to drift of the composition over extended cultivation periods. This adds more constraints and less flexibility to the manufacturing process compared to that of a monoclonal protein manufacturing process, and may result in a higher risk of manufacturing failure, for example in cases of technical problems with the production reactors leading to delays.

In the case of batch or fed batch manufacturing, which is the preferred technology for manufacturing of recombinant antibodies, the majority of the population doublings actually take place before inoculation of the production reactor. A typical upstream process may with respect to population doublings look like this: To generate a pWCB by expansion of a vial from a pMCB, 8-12 population doublings are needed. The seed train expansion will typically require 10-20 doublings depending of the final manufacturing scale, whereas the number of population doublings taking place in the inoculum reactor typically will be 2-5 generations. In the fed batch production reactor, the cells go through a relatively low number of generations, such as 5-8. In total, about 25-43 generations are needed to complete the upstream process when using a single-batch manufacturing process starting from a polyclonal master cell bank. Of these, about 20-37 population doublings occur before inoculation of the production vessel. It will be apparent that the shorter the mixed cultivation period is, the more feasible it is to control and maintain a desired compositional ratio. Thus, for polyclonal products where it is difficult to obtain similar growth rates or where it is critical to obtain a compositional ratio of very narrow limits, it will be advantageous to maintain the individual cell lines in separate containers during the initial phases of the manufacturing process and mix the cells at a late point in time.

Recombinant protein products are typically expressed in vitro using isolated cells expressing a heterologous or endogenous protein product by culturing these cells in liquid or semisolid medium under conditions favouring expression of the protein product. These methods are particularly advantageous for expression of secreted protein products as these can easily be separated from the cells as a first step of the protein purification.

The containers used for culturing may e.g. be selected from the group consisting of shaker flasks, roller bottles, T-flasks and disposable or non-disposable bioreactors. In preferred embodiments the containers comprise one or more disposable containers. Disposable containers may include so-called Wave Bags that are available from a number of manufacturers in different sizes ranging from 10 L and up to more than 1000 litres. Suitable disposable bioreactors include but are not limited to BIOSTAT® CultiBag (Sartorius Stedim Biotech), Cell Maker Lite2™ (Cellexus Biosystems), Biowave® (Wave Biotech AG), Wave Biotech LLC (GE Healthcare) bags, Single-Use Bioreactors S.U.B. (Hyclone Thermo Fisher Scientific) and FlexFactory™ (Xcellerex). As the present invention involves the use of separate bioreactors for distinct members of the polyclonal protein either in parallel or serial manufacturing for at least part of the upstream process, it is an advantage to use the relatively inexpensive disposable bioreactors instead of costly steel tanks. In addition, the disposable bioreactors add flexibility to the methods of the invention as it is easier and less expensive to increase the number of bioreactors compared to using multiple use steel tanks.

While methods have been developed for expression of different members of a polyclonal protein, these methods are in some cases inadequate. If for example the polyclonal protein comprises different protein members that cannot be purified using the same downstream process procedure, then expressing the distinct members in a single batch is no advantage. Also, if for some reason one wishes to express one distinct member in a particular cell type and other members in another cell type, then single batch manufacturing is less preferable.

Finally, there may be cases where the difference in expression level and/or growth rate of different cell clones expressing different members of a polyclonal protein is so large that combining the different clones in a polyclonal cell bank is disadvantageous.

Separate Seed Train

One embodiment of the present invention relies on having different monoclonal cell banks that are thawed in separate culture containers, such that optimal thawing and adaptation conditions can be used for each clone expressing a distinct member of the polyclonal protein. During these initial steps of thawing and adaptation the viability of the cells is under pressure, and even minor differences in the survival among clones in these early steps may have a dramatic effect on the relative distribution of protein members in the final product.

According to this embodiment of the invention individual producer cell lines expressing the different members of the polyclonal protein are kept separate during thawing, adaptation and seed train expansion. The clones can then be mixed according to desired criteria and be cultured in one single batch for the inoculum train, production phase, harvest and downstream processing.

Separate Inoculum Train

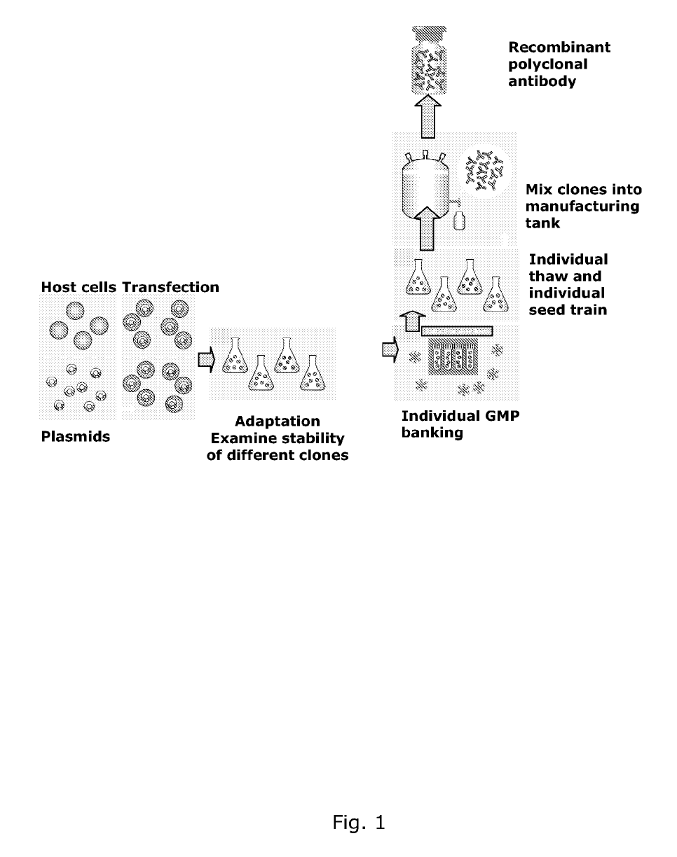

In a further embodiment the different clones expressing different distinct members of the polyclonal protein are kept separate up to and including the inoculum train (FIG. 1). According to this embodiment, it is possible to have the same cell number for each individual producer cell line at the start of production phase. As mentioned above, the polyclonal phase is relatively short in this embodiment, with a limited number of cell divisions, so that any differences in growth rate among the cell clones will have limited effect on the composition. In this way it is easier to combine different individual producer cell lines to obtain a predetermined distribution of the distinct members of the polyclonal protein at the end of the production phase.

An important advantage of using a separate seed train, and in particular of using a separate seed train and separate inoculum train, is that the number of generations from the point at which the separate clones are pooled and until the end of the production phase is reduced. As a result, the total number of generations in which different cell clones are pooled and cultured together is minimized. This allows for a greater overall stability and uniformity in the final product from batch to batch, and thus a greater degree of control over the final result, since there are fewer generations in which one or more clones have the possibility to outgrow or outproduce other clones in the mixture of different cell clones.

Another advantage of this approach compared to e.g. use of a polyclonal master cell bank is that it allows greater flexibility in adapting the polyclonal mixture as needed. For example, if one of the cell clones in a polyclonal MCB is found to be unstable or otherwise suboptimal, the entire polyclonal MCB would have to be recreated. In contrast, the present invention allows a single suboptimal clone to be removed or alternatively replaced by a new clone that produces the same protein with less time and effort. It also makes it possible to relatively easily add a new clone that produces a new protein to an existing mixture of clones producing an established polyclonal protein. In addition, this approach makes it easier to upscale the production phase while minimizing the risk of changes in the final protein composition, for example using Wave Bags or other disposable bioreactors for the seed and inoculum train.

A still further advantage is the possibility to move more quickly into pre-clinical development of a polyclonal antibody or other therapeutic protein once desired distinct members of the polyclonal protein have been identified. This is because once suitable individual protein members have been identified, it is only necessary to test for stability of the individual clones, which can then be used directly for production purposes, i.e. without also having to spend additional time on stability studies of a polyclonal master cell bank and polyclonal working cell bank. Furthermore, for pre-clinical and clinical phase I/II testing it may also be possible to perform the manufacturing based on MCBs without having to spend time on generation and testing of WCBs.

Separate Production Phase

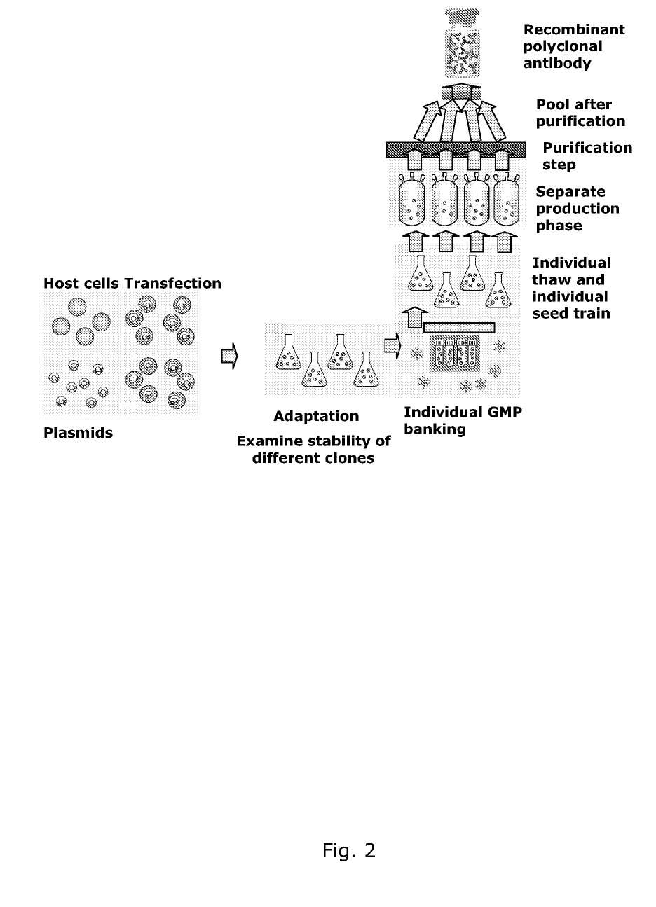

In a still further embodiment, the production phase is also carried out separately for distinct members of the polyclonal protein (FIG. 2). This allows for expression of different members in different cell types and/or under different conditions and also allows for situations where the complete downstream processing cannot be carried out for all distinct members of the polyclonal protein in one and the same procedure. This scenario also provides full freedom with respect to selecting expression platforms for the distinct members of the polyclonal protein.

Thus, for example, one member can be expressed in yeast cells and one can be expressed in mammalian cells. Alternatively or additionally, one protein member can for example be expressed in isolated plant cells in vitro and one can be expressed in bacteria if so desired. It is known in the art that different species result in different post-translational modifications of the expressed protein. In this way a product with such different post-translational modifications can be manufactured and subjected to one common or partly common downstream processing.

This manufacturing scenario also enables the use of different cultivation modes for different members of the polyclonal protein. One member may be manufactured using a batch process, while other members may be manufactured using a fed-batch or perfusion process.

A further advantage of having a separate production phase for distinct members of the polyclonal protein is that the distinct protein members can be mixed or combined prior to downstream processing in a pre-determined ratio. This reduces any skewed distribution that could potentially be caused by differences in proliferation rate and/or expression levels.

A further advantage is that serial manufacturing of the distinct protein members can be performed. This allows for manufacturing of a complex polyclonal protein with many distinct protein members in one or a few bioreactors. The expressed distinct protein members can be harvested following expression one after the other or a few at a time and stored until all members have been expressed. The polyclonal protein may then be purified using one downstream processing procedure.

Separate Harvest

Following separate expression of the distinct protein members, the subsequent harvest step may be performed separately for one or more members of the polyclonal protein, or two or more members may be harvested using a common procedure.

Expressed protein product may be obtained by collecting supernatant or cells from different bioreactors, or in the case of production in plants or animals, by isolating the protein from harvested plants or from e.g. milk or eggs of transgenic animals.

Separate First Purification Step

The first phase of the downstream process (DSP) typically includes one or more steps to separate the protein product from the cells and cell debris (cell walls, membranes and fragments). This may be done using procedures for clarification, including but not limited to centrifugation and filtration. When the distinct members are manufactured separately, the initial clarification may be carried out separately for the distinct members or jointly for two or more of the members, although in cases of separate manufacturing and harvest of the distinct proteins the separately harvested supernatants will typically also be clarified separately.

Following clarification, the next step may include an affinity chromatography step, such as Protein A or Protein G purification carried out in a bind and elute mode (catch mode). Such a purification step is typically very efficient at getting rid of the vast majority of contaminants (host cell protein, DNA, virus, medium ingredients). As proteases and other enzymes may constitute part of the contaminants, getting rid of these at an early stage is important for the stability of the protein product.

In addition, an initial affinity chromatography step typically results in a significant reduction in volume, making it easier and more convenient to store the partially purified protein product following this purification step. In the case of serial production, it is an advantage to perform a volume reduction step immediately or shortly after harvesting the protein. Also, for reasons of stability of the protein product, it is an advantage to remove the major part of the contaminants as quickly as possible following protein harvest. Frozen aliquots each comprising one distinct member of a polyclonal protein can subsequently be thawed, mixed and subjected to further DSP.

A separate initial chromatography step can also be applied for at least one of the distinct members if e.g. the distinct members of the polyclonal protein include antibodies with different isotypes that cannot be captured on the same column, or if the production of the individual members is separated in time. For example, the polyclonal protein may be a polyclonal antibody comprising at least one distinct antibody member that can be purified on a Protein A column and at least one other distinct antibody member that can be purified on a Protein G column. The remaining part of downstream processing may be carried out as one process.

Separate Further Chromatography Steps

In other embodiments, further downstream processing may also be carried out separately for distinct protein members. This may be advantageous if very different recovery rates are expected for a particular purification step and/or if the distinct members differ so much with respect to size, charge and/or hydrophobicity that one common purification procedure cannot be used. The presence of very different contaminants for different distinct protein members may also justify the use of separate chromatography steps.

This embodiment allows for optimisation of the steps for each distinct protein member to maintain high recovery rates for all protein members. This may result in less loss during the downstream processing.

Separate Preparation of Drug Substance

In a still further embodiment of the invention all steps up to obtaining the “drug substance” are carried out separately for distinct protein members. In this scenario the drug substance comprising different members of the polyclonal protein may be manufactured in parallel or serially. This makes it possible to combine distinct protein members in a precise pre-determined ratio. This may be an advantage if the precise ratio is important for function. The complete upstream and downstream processing is separate and can be optimised for each distinct protein member. This may lead to higher expression levels and recovery rates. Using this embodiment of the invention, it is possible to derivatise one or more members of the protein product, e.g. to link a toxin, a polymer, or another non-protein group to one or more but not all of the distinct protein members. Such derivatisation requires that the protein has been purified prior to derivatisation. In addition, derivatisation has the result that the derivatised product cannot be easily purified together with the other distinct non-derivatised protein members. Separate characterisation and/or release assays for each distinct member of the polyclonal protein can be carried out individually for each of the drug substances comprising a distinct member of the polyclonal protein. If one or more of the distinct members are derivatised, it may be an advantage to be able to carry out particular release or characterisation assays on this distinct member alone.

The obtained drug substance(s) is/are finally formulated into a drug product together with the required pharmaceutical excipients, carriers etc.

Specific Manufacturing Scenarios

In the following, different manufacturing scenarios for production of an antibody product according to the invention are described stepwise. These are provided as non-limiting examples.

1) Partly Separate Seed Trains and Mixing Before Production

This scenario includes the following steps (refer to FIG. 1):
transfection in pools
selection
screening for high-producing clones
separate growth to a certain stage of seed/inoculum train
mixing
single-batch production of polyclonal product.

The scenario initially includes providing a set of expression vectors encoding each of the distinct polyclonal protein members. Each of the expression vectors is transfected into the genome of suitable host cells separately, so that one cell expresses only one member of the polyclonal protein. Selection of transfected clones may e.g. be performed using a co-expressed dominant genetic marker such as a drug resistance marker.

High producing single cell clones can be selected using protocols for high throughput screening. Clones are further screened for appropriate bioreactor parameters and for cell specific productivity and maximum titers.

The clones encoding each of the distinct polyclonal protein members may subsequently be aligned into groups using data on bioreactor parameters, productivity and titers according to an algorithm for polyclonal production. The best producing set (i.e. balancing productivity and compositional stability) is chosen. Alternatively, experiments may be performed to test for the combinations of clones providing the best co-culture results.

For each distinct polyclonal protein member, a master cell bank (MCB) is generated. Each MCB is then used in a separate seed train. The separate seed trains are then used to inoculate a bioreactor according to specified criteria based on data from the characterization steps or based on experimental results. The inoculum is then used in single batch production in one bioreactor and the harvested polyclonal protein is subjected to a single downstream process.

2) Separate Production in Individual Producer Cell Lines, Mixing Before Purification.

This scenario includes the following steps (refer to FIG. 2):
transfection of cells in pools
selection of transfectants
screening for high-producing cell lines/clones
separate production and mixing before purification

The scenario initially includes providing a set of expression vectors encoding each of the distinct polyclonal protein members. Each of the expression vectors is transfected into the genome of suitable host cells separately so that one cell expresses only one member of the polyclonal protein. Subsequently, selection for a co-expressed dominant genetic marker such as a drug resistance marker is carried out to select transfected clones.

High producing single cell clones can be selected using protocols for high throughput screening. Clones are further screened for appropriate bioreactor parameters and for cell specific productivity and maximum titers.

The best clones encoding each of the distinct polyclonal protein members are chosen and a master cell bank, MCB, is generated for each distinct polyclonal protein member. Each MCB is then used in traditional upstream production. The supernatants from all bioreactor runs for each distinct polyclonal protein member are collected in hold tanks/containers and mixed, and the mixed polyclonal supernatant is then purified. Optionally, a volume reduction step can be carried out prior to collection in hold tanks. Volume reduction steps may comprise ultrafiltration or affinity chromatography, e.g. Protein A chromatography. The volume reduction step is carried out separately for distinct protein members.

3) Separate Production and Purification, Mixing of Purified Proteins

This scenario includes the following steps:
transfection in pools
selection
screening for high-producing clones
separate production
separate purification
mixing of purified antibodies (drug substances)

The scenario is identical to scenario 2 until the end of the upstream process. The distinct members of the polyclonal protein are then subjected to traditional separate downstream processing. The purified drug substances from all runs for each distinct polyclonal protein member are collected and optionally stored and mixed according to the specified criteria to obtain the final drug product.

Mixing of Cells and Proteins

Whenever different populations of cells are mixed during the upstream processing part of the manufacturing, this will affect the composition of the polyclonal protein obtained as the end product.

One approach for mixing cells includes mixing or combining equal numbers of cells of the at least two different populations of cells. However, if different populations of cells have different expression levels or different growth rates, this may not lead to an equal distribution of the distinct protein members in the end product. In order to compensate for this, different numbers of cells of the at least two different populations of cells may be combined. Cells from different populations may thus be combined to obtain a polyclonal cell population capable of expressing approximately equal amounts of the distinct members of the polyclonal protein, such as mixing them in a pre-defined ratio, which may be determined experimentally.

In other embodiments, the cells from the at least two different populations of cells are combined in a ratio giving a pre-defined ratio of the distinct protein members in the drug substance or drug product. This may be done by using knowledge about the expression levels and/or growth rate of the different populations of cells and/or knowledge about the recovery of the distinct members in the at least one purification step to calculate the ratio prior to combining the cells. Alternatively, mix ratios suitable for obtaining a desired result may be determined empirically based on experimental data.

When combination is carried out at the end of upstream processing or during downstream processing this may be done simply by combining equal volumes from cell cultures of the distinct members of the polyclonal protein. Alternatively, equal quantities of the distinct members of the polyclonal protein may be combined if such information has been obtained.

The at least two distinct members of the polyclonal protein may be combined in a ratio giving a pre-defined ratio of the distinct protein members in the drug substance or drug product. This may be done by using knowledge about recovery of the distinct members in the at least one purification step to calculate the ratio prior to combining.

Polyclonal Proteins

In most embodiments of the invention, the polyclonal protein is not naturally associated with the cells expressing the protein members, and the expression system is recombinant.

The present invention provides methods for the consistent manufacturing of recombinant polyclonal proteins that are preferably secreted and more preferably selected from the immunoglobulin superfamily, a family of proteins with immunoglobulin-like domains. Most of the members of the immunoglobulin superfamily are involved in cell surface recognition events. Sequence analysis suggests that antibodies, T cell receptors, MHC molecules, some cell adhesion molecules and cytokine receptors are highly homologous. Especially members of this family that contain variable regions are suitable for the generation of recombinant polyclonal proteins according to the present invention. Such members include antibodies, membrane bound antibodies (B cell receptors), Fab fragments, Fv fragments, single chain Fv (scFv) fragments, T cell Receptors (TcRs), soluble TcRs, TcR variable domain fragments, TcR variable domain fragments linked by a polypeptide linker, and other antibody or TcR derived fragments. In particular, it is contemplated that the present invention can be used for large-scale manufacturing and production of recombinant therapeutic polyclonal antibodies or antibody fragments and TcRs and TcR fragments. In a preferred embodiment, the polyclonal protein is a polyclonal antibody or polyclonal antibody fragment.

The recombinant polyclonal protein of the present invention refers to a protein composition comprising different, but homologous protein molecules, where the differences e.g. reflect a naturally occurring diversity. Thus, each protein molecule is homologous to the other molecules of the composition, but also contains one or more stretches of variable polypeptide sequence characterized by differences in the amino acid sequence between the individual members of the polyclonal protein. The differences in the amino acid sequences that constitute the variable polypeptide sequence might be as little as one amino acid but will normally constitute more than one amino acid. The natural variability of a polyclonal antibody or TcR is generally located in the so-called variable regions or V-regions of the polypeptide chains.

In one embodiment of the present invention individual members in a polyclonal protein comprise variable regions that are between approximately 80 and 120 amino acids long. The variable regions may comprise hyper-variable domains, e.g. complementarity determining regions (CDRs). In naturally occurring TcRs there are four CDRs in each variable region. In naturally occurring antibodies there are three CDRs in the heavy chain and three CDRs in the light chain.

In an additional embodiment of the present invention the variable regions of the individual members of a polyclonal protein comprise at least one hyper-variable domain that is between 1 and 26 amino acids long, preferably between 4 and 16 amino acids long. This hyper-variable domain can correspond to a CDR3 region. For antibodies, each variable region preferably includes three hyper-variable domains. These can correspond to CDR1, CDR2 and CDR3. For TcRs each variable region preferably constitutes four hyper-variable domains. These can correspond to CDR1, CDR2, CDR3 and CDR4. The hyper-variable domains may alone constitute the variable sequences within a variable region of a recombinant polyclonal protein of the present invention.

In the context of the present invention, variability in the polypeptide sequence (the polyclonality) can also be understood to describe differences between the individual antibody molecules residing in so-called constant regions or C regions of the antibody polypeptide chains, e.g., as in the case of mixtures of antibodies containing two or more different antibody isotypes, such as the human isotypes IgG1, IgG2, IgG3, IgG4, IgA1, IgA2, IgM, IgD, and IgE, or the murine isotypes IgG1, IgG2a, IgG2b, IgG3, IgM, and IgA. Thus, a recombinant polyclonal antibody may comprise antibody molecules that are characterized by sequence differences between the individual antibody molecules in the variable region (V region) or in the constant region (C region) or both. Preferably, the antibodies are of the same isotype, as this eases the subsequent purification and characterization considerably. It is also conceivable to combine antibodies of different isotypes or, preferably, different subclasses. For example, antibodies of isotype IgG1, IgG2 and IgG4 may be combined, as these can all be purified together using Protein A affinity chromatography. In the case of antibodies of different isotypes or subclasses, the polyclonality can occur in the constant part or in the variable domain, or both. In a preferred embodiment, all antibodies constituting the polyclonal antibody have the same constant region to further facilitate purification. More preferably, the antibodies have the same constant region of the heavy chain. The constant region of the light chain may also be the same across distinct antibodies. In one embodiment, the at least two populations of cells are thus identical except for differences in expression vector sequences that encode the distinct protein members. The at least two cell populations may, for example, be identical except for differences in at least one expression vector sequence that encodes a variable region of the distinct protein members.

In a preferred embodiment of the invention, the recombinant polyclonal protein is a recombinant polyclonal antibody or antibody fragment. In another preferred embodiment of the invention, the recombinant polyclonal protein is a recombinant polyclonal TcR or TcR fragment.

Polyclonality in the so-called constant region, particularly the heavy chain of the antibodies, is of interest with regard to therapeutic application of antibodies. The various immunoglobulin isotypes have different biological functions (summarized in Table 1), which might be desirable to combine when utilizing antibodies for treatment, because different isotypes of immunoglobulin may be implicated in different aspects of natural immune responses (Canfield and Morrison 1991. J. Exp. Med. 173, 1483-91; Kumpel et al. 2002. Transfus. Clin. Biol. 9, 45.-53; Stirnadel et al. 2000. Epidemiol. Infect. 124, 153-162).

TABLE 1
Biological functions of the human immunoglobulin isotypes

| | Human Immunoglobulin | | | | | | | | |
|---|---|---|---|---|---|---|---|---|---|
| | IgG₁ | IgG₂ | IgG₃ | IgG₄ | IgA₁ | IgA₂ | IgM | IgD | IgE |
| Classical | +++ | ++ | ++++ | + | − | − | ++++ | − | − |
| complement | | | | | | | | | |
| activation | | | | | | | | | |
| Alternate | + | + | + | +++ | + | − | − | + | − |
| complement | | | | | | | | | |
| activation | | | | | | | | | |
| Placental | + | ++ | + | ++ | − | − | − | − | − |
| transfer | | | | | | | | | |
| Bacterial | + | + | + | + | +++ | +++ | + | ? | ? |
| lysis | | | | | | | | | |
| Macrophage/ | + | − | + | + | + | + | − | − | − |
| other | | | | | | | | | |
| phagocytes | | | | | | | | | |
| binding | | | | | | | | | |
| Mast cell/ | − | − | − | − | − | − | − | − | − |
| basophils | | | | | | | | | |
| binding | | | | | | | | | |
| Staphyl- | + | + | − | + | − | − | − | − | − |
| ococcal | | | | | | | | | |
| Protein A | | | | | | | | | |
| reactivity | | | | | | | | | |

As described above, polyclonality may reside in the constant part, so that at least one distinct member of the polyclonal protein may comprise one constant region, and at least one other distinct member comprises a different constant region. It may also be preferable to have different glycosylation patterns on the expressed proteins, which may be obtained through the use of different host cells for distinct members of the polyclonal protein. At least one distinct member of the polyclonal protein may thus comprise one glycosylation pattern, while at least one other distinct member comprises a different glycosylation pattern.

In other embodiments, polyclonality is obtained through derivatisation of one or more of the distinct members of the polyclonal protein. For example at least one distinct member of the polyclonal protein may be derivatised using a chemical method for modification of the protein such as coupling to a toxin and at least one other distinct member is not derivatised. Different types of chemical derivatisation may be employed for different distinct members of the polyclonal protein.

The polyclonal protein may comprise at least three distinct members, such as at least 4 distinct members, for example at least 5 distinct members, such as at least 6 distinct members, for example at least 7 distinct members, such as at least 8 distinct members, for example at least 9 distinct members, such as at least 10 distinct members, for example at least 15 distinct members, such as at least 20 distinct members, for example at least 25 distinct members. For most purposes the number of distinct members in the polyclonal protein is less than 50, such as less than 45, for example less than 40, such as less than 35, for example less than 30, such as less than 25, for example less than 20, such as less than 15, for example less than 10, such as less than 5.

In the different manufacturing scenarios described above it is to be understood that the separate phase of the manufacturing may be carried out for each of the distinct members, e.g. so that all distinct members are expressed individually. However, it is also conceivable that only one, two or three distinct members are manufactured separately, while other members are manufactured in a single batch or in a few single batches.

The Host Cell

Host cells can be generated from any cell which can integrate DNA into its chromosomes or retain extra-chromosomal elements such as plasmids, mini-chromosomes, YACs (Yeast artificial chromosomes), MACs (Mouse artificial chromosomes), or HACs (Human artificial chromosomes). MACs and HACs are described in detail in WO 97/40183.

The host cells may be prokaryotic or eukaryotic, and in some cases prokaryotic cells may be used for expression of one or more of the distinct members and eukaryotic cells may be used for one or more other distinct members.

The eukaryotic cells may be from a eukaryotic organism selected from the group consisting of plants, yeast, fungi, vertebrates and invertebrates. Alternatively, the eukaryotic cells may also be a hybridoma or immortalised B-cells for expression of antibodies.

Preferably, mammalian cells such as CHO cells, COS cells, BHK cells, myeloma cells (e.g., Sp2/0, YB2/0 or NS0 cells), fibroblasts such as NIH 3T3, immortalized human cells such as HeLa cells, HEK 293 cells or PER.C6 cells, are used. However, non-mammalian eukaryotic or prokaryotic cells, such as plant cells, insect cells, yeast cells, fungi, bacteria such as E. coli etc., can also be employed. In a preferred embodiment, the host cell is a mammalian cell such as a CHO cell.

In one embodiment of the present invention, the cell line which is to be used as starting material is sub-cloned by performing limiting dilution of the cell line down to the single cell level, followed by growing each single cell to a new population of cells prior to transfection with a library of vectors of interest. Such sub-cloning can also be performed later in the process of selecting the right cell line, if desired. Other methods for single cell cloning include: FACS cloning (Brezinsky et al. J. 2003. Immunol Methods 277, 141-155), LEAP™ technology (from Cyntellect, San Diego, Calif., USA), and ClonePix (from Genetix, UK). In this manner, a particular population of cells is derived from one cloned cell expressing one distinct member of the polyclonal protein.

Other manufacturing systems can be used in addition to traditional recombinant manufacturing in isolated mammalian cells and bacteria.

For example, antibodies can be expressed recombinantly in fungi, yeast and intact plants, in transgenic birds (recovering the antibody from the eggs) and in transgenic mammals (recovering the antibody from the milk).

Methods for manufacture of antibodies in fungi are known in the art, e.g. as described in WO 2005/070962; Nyyssonen et al. 1993, Nat Biotech 11:591-5; and in Ward et al. 2004, Appl. Environ. Microbiol. 70:2567-76.

Methods for manufacture of monoclonal antibodies in yeast are known in the art, e.g. as described in Li et al., 2006, Nature Biotech. 24(2):219-215.

Methods for manufacture of monoclonal antibodies in intact plants are known in the art, e.g. as described in Bouquin et al., 2002, Transgenic Res. 11:115-22; Hood et al., 2002, Curr. Opin. Biotechnol. 13:630-5; and in Stoger et al., 2004, Methods Mol. Biol. 248:301-318.

Methods for manufacture of monoclonal antibodies in birds, where the antibody product is found in the egg, are known in the art, e.g. as described in Zhu et al., 2005, Nature Biotech. 23:1159-69.

Antibodies can also be manufactured recombinantly in transgenic mammals, where the recombinant antibody product can be found in the milk. Examples of such methods are described in the following references: Behboodi et al., 2005, Cloning Stem Cells 7:107-18; Hodges et al., 2003, Reprod. Biol. Endocrinol. 1:81; Houdebine L M. 2002, Curr Opin. Biotechnol 13:625-9; Jang et al., 2006, Theriogenology 65:1800-12; Lai et al., 2003, Reprod. Biol. Endocrinol. 1:82; Lonberg N. 2005, Nature Biotech. 23:1117-25; Santora et al., 2006, Biomed. Chromatogr. 20:843-56; Tang et al., 2008, Transgenic Res; and Young et al., 1998, Res Immunol 149:609-10.

The Vector for Integration

A suitable vector comprises a suitable selection gene. Suitable selection genes for use in mammalian cell expression include, but are not limited to, genes enabling nutritional selection, such as the thymidine kinase gene (TK), glutamine synthetase gene (GS), tryptophan synthase gene (trpB) or histidinol dehydrogenese gene (hisD). Selection markers that may be used include antimetabolite resistance genes conferring drug resistance, such as the dihydrofolate reductase gene (dhfr), which can be selected for with hypoxanthine and thymidine deficient medium and further selected for with methotrexate, the xanthine-guanine phosphoribosyltransferase gene (gpt), which can be selected for with mycophenolic acid, the neomycin phosphotransferase gene (neo) which can be selected for with G418 in eukaryotic cells and neomycin or kanamycin in prokaryotic cells, the hygromycin B phosphotransferase (hyg, hph, hpt) gene, which can be selected for with hygromycin, the puromycin N-acetyl-transferase gene (pac), which can be selected with puromycin, or the Blasticidin S deaminase gene(Bsd), which can be selected with blasticidin, and the Zeocin resistance gene (Sh ble), which mediates resistance towards Zeocin and Bleomycin. Finally, genes encoding proteins that enable sorting e.g. by flow cytometry can also be used as selection markers, such as green fluorescent protein (GFP), the nerve growth factor receptor (NGFR) or other membrane proteins, or beta-galactosidase (LacZ).

Preferably, the selectable marker encodes a gene product for which the host cell is deficient, which avoids the addition of e.g. an antibiotic to the culture medium. In another preferred embodiment cells are continuously cultured under conditions favoring growth of cells expressing the selectable marker. This is particularly useful when the selectable marker is a gene product in which the host cell is deficient, as it allows the use of selection conditions throughout the cultivation period without the addition of e.g. antibiotics.

In the case of expression of dimeric or multimeric proteins, one expression vector may encode all subunits of a distinct polyclonal protein member. Alternatively, the expression vectors may include two or more subsets of expression vectors, where a first subset comprises variant nucleic acid sequences encoding one subunit of the protein, and a second subset comprises variant nucleic acid sequences encoding another subunit of the protein, such that each transfection is performed with a member from the first subset and a member for the second subset of expression vectors. In the case of antibodies, the expression vectors may be constituted by two subsets of expression vectors, where the first subset comprises variant nucleic acid sequences encoding an antibody heavy chain, and the second subset comprises variant nucleic acid sequences encoding an antibody light chain, such that each transfection is performed with a member from the first subset and a member for the second subset of expression vectors.

The selection marker may be located on a separate expression vector, so that co-transfection is performed with an expression vector coding for the selection marker and one or more expression vectors coding for the protein of interest or subunits of the protein of interest. The selection marker may also be located on the expression vector coding for the protein of interest. In this latter case, the selection marker is preferably located on a transcript which also encodes the protein of interest or one of its subunits. This can be done e.g. using an IRES construct. In the case of a multimeric protein, such as a multimeric antibody, the selection marker is preferably located on the transcript which encodes the largest subunit, for example the heavy chain of an antibody.

The vector for integration of the gene of interest further comprises DNA encoding one member of the recombinant polyclonal protein of interest, preceded by its own promoter directing expression of the protein, e.g. a mammalian promoter for expression in mammalian cells. If a member of the recombinant polyclonal protein of interest comprises more than one protein chain, e.g. if the member is an antibody or T cell receptor, the DNA encoding the individual chains of the protein can be preceded by their own promoter directing high levels of expression (bi-directional or uni-directional) of each of the chains. In a bi-directional expression a head-to-head promoter configuration in the expression vector can be used, and for a uni-directional expression two promoters or one promoter combined with e.g. an IRES sequence can be used for expression. A bi-cistronic expression vector with two different subunits encoded by the same transcript and separated by an IRES sequence is likewise conceivable.

Suitable head-to-head promoter configurations are, for example, the AdMLP promoter together with the mouse metallothionein-1 promoter in both orientations, the AdMLP promoter together with the elongation factor-1 promoter in both orientations, the CMV promoter together with the MPSV promoter in both orientations, or the CMV promoter used in both orientations.

In the case of antibodies, experience has shown that the amount of heavy chain expressed by a cell should not exceed the amount of light chain. Therefore, the promoter directing expression of the light chain is preferably at least as strong as the promoter directing expression of the heavy chain.

A nucleic acid sequence encoding a functional leader sequence can be included in the expression vector to direct the gene product to the endoplasmic reticulum or a specific location within the cell such as an organelle. A strong polyadenylation signal can be situated 3′ of the protein-encoding DNA sequence. The polyadenylation signal ensures termination and polyadenylation of the nascent RNA transcript and is correlated with message stability. The DNA encoding a member of the recombinant polyclonal protein of interest can, for example, encode both the heavy and light chains of an antibody or antibody fragments, each gene sequence optionally being preceded by its own mammalian promoter elements and/or followed by strong poly A signals directing high level expression of each of the two chains.

The expression vector for integration can carry additional transcriptional regulatory elements, such as enhancers, anti-repressors, or UCOE (ubiquitous chromatin opening elements) for increased expression at the site of integration. Enhancers are nucleic acid sequences that interact specifically with nuclear proteins involved in transcription. The UCOE opens chromatin or maintains chromatin in an open state and facilitates reproducible expression of an operably-linked gene (described in more detail in WO 00/05393 and Benton et al., Cytotechnology 38:43-46, 2002). Further enhancers include Matrix Attachment Regions (MARs) as described e.g. in Girod & Mermod 2003 (“Chapter 10: Use of scaffold/matrix-attachment regions for protein production”, pp 359-379 in Gene Transfer and Expression in Mammalian Cells, SC Makrides (ed.), 2003, Elsevier Science BV). Anti-repressor elements include but are not limited to STAR elements (Kwaks et al., Nat Biotechnol. 2003 May; 21(5):553-8). When one or more of the regulatory elements described in the above are integrated into the chromosome of a host cell they are termed heterologous regulatory elements.

If expression levels need to be increased, gene amplification can be performed using selection for a DHFR gene or a glutamine synthetase (GS) gene, a hprt (hypoxanthin phosphoribosyltransferase) or a tryptophan synthetase gene. This requires the use of vectors comprising such a selection marker.

Culturing of the Producer Cells

During the culturing step, the transformed cells are cultured by preparing and cultivating an inoculum (the seed train), scaling up the inoculum in a single bioreactor or a series of bioreactors (the inoculum train), and producing and accumulating protein from the inoculum (the production phase).

In the seed train phase, the transformed cells from the transforming step are recovered into an inoculum cultivation medium to create an inoculum. The transformed host cells are cultured by methods known in the art in a liquid medium containing assimilable sources of carbon (carbohydrates such as glucose or lactose), nitrogen (amino acids, peptides, proteins or their degradation products such as peptones, ammonium salts or the like), and inorganic salts (sulfates, phosphates and/or carbonates of sodium, potassium, magnesium and calcium). The inoculum cultivation medium preferably includes a conventional nutrient medium such as Dulbecco's Modified Eagle's Medium (DMEM) (Sigma), Ham's F10 (Sigma), Minimal Essential Medium (MEM) (Sigma), RPMI-1640 (Sigma) or NCTC-135. The cultivation medium is preferably a serum-free medium, and more preferably a medium free of animal proteins such as EX-CELL® 302 (SAFC Biosciences), still more preferably a protein-free medium such as EX-CELL® 325 (SAFC Biosciences), and most preferably a chemically defined medium such as OptiCHO™ (Invitrogen), or ProCHO4™ or PowerCHO2-CD™ (Lonza BioWhittaker).

Any of these media can be supplemented as necessary with amino acids (glutamine), hormones or other growth factors (insulin, transferrin, or epidermal growth factor), vitamins, salts (zinc sulfate, sodium chloride, phosphate), buffers, nucleotides, antibiotics, ionic surfactants, and/or glucose or an equivalent energy source. The medium can further contain trace elements that are growth promoting substances, such as iron chelates (e.g., chelate B, Invitrogen Corp., Carlsbad, Calif.), and/or manganese. During the seed train, culture conditions, such as temperature, pH, and the like, are monitored to ensure rapid cell growth.

During the inoculum train, the inoculum is scaled-up in scale-up medium through sequential steps of cultivation. Such steps can be performed in any suitable container, including cell culture flasks, stir bottles, roller bottles, rotary bioreactors, and spinner flasks. Preferably, the inoculum train is carried out in a bioreactor, so that cells can be transferred to a production reactor through a closed tubing system. The scale-up medium also includes a conventional nutrient medium and can include amino acids supplied by hydrolysates (e.g., HySoy®, Quest International, Chicago, Ill.), hormones or other growth factors, vitamins, salts, buffers, nucleotides, antibiotics, ionic surfactants, iron chelates, and glucose or an equivalent energy source. During the inoculum train in bioreactors, the pH, oxygen saturation and waste products of the inoculum are monitored.

During the production phase, the cells are transferred to a stir tank or airlift bioreactor or a disposable or single-use bioreactor and fed with a complex growth medium containing sugars, amino acids, salts, trace elements and growth factors, which are combined in such quantities so as to maintain the pH, osmolality, and other essential parameters of the growth medium for consistent, robust, rapid cell growth. Examples of such commercially available feed solutions are the Cell Boost 1-6 (Hyclone) and EfficientFeed™ A and B (Invitrogen). Alternatively, custom made feed solutions matching the needs of the particular production cells can be used. The use of osmoprotectant compounds, such as betaine or proline, for example, can protect cells from osmotic stress while enhancing antibody productivity. The temperature, dissolved oxygen, pH, pressure, gas flow rate and stir rate are also controlled during the production phase. During the production phase, the cells express the protein internally or secrete the protein into the surrounding medium. Those cells that express protein within their structures can be chemically or mechanically fragmented in order to harvest the protein. More complex cells such as mammalian cells can produce glycosylated cellular products and secrete the protein into the cell culture medium for isolation.

Harvesting and Purification

During the harvesting step, the protein is removed from the cell culture by any means known in the art. For example, when the proteins are produced intracellularly by the transformed cells, centrifugation or ultrafiltration can be used to remove the host cells or lysed cells. Where the protein is secreted into the medium, the protein can be removed from the mixture of compounds fed to the cells and from the by-products of the cells themselves by using commercially available protein concentration filters, for example Amicon® or Millipore Pellicon® ultrafiltration units.

During the purifying step, the proteins are subjected to one or more purification steps, including various chromatography methods. Examples of such purification procedures include anion exchange chromatography and cation exchange chromatography, as well as various filtration methods, such as tangential flow filtration using Pellicon® membranes (Millipore, Billerica, Mass.), nanofiltration using DVSO filters (Pall Corporation, East Hills, N.Y.), for example to reduce potential viral contamination, and appropriate size dead end filtration (such as 0.45 μm and 0.2 μm filters), fractionation using hydrophobic interaction chromatography (e.g. on phenyl sepharose), ethanol precipitation, isoelectric focusing, Reverse Phase HPLC, chromatography on silica, chromatography on Heparin Sepharose™, chromatofocusing, SDS-PAGE, ammonium sulfate precipitation, hydroxylapatite chromatography, gel electrophoresis, dialysis, and affinity chromatography (e.g., using protein A, protein G, an antibody, a specific substrate, ligand or antigen as the capture reagent).

The proteins of the present invention can also be modified or derivatised.

Examples of such modification include post-translation modifications, such as glycosylation (both O-linked and N-linked), acetylation, phosphorylation, ubiquitination, polymer conjugation, and the like. Some of these modifications can be carried out in vivo using the host cell machinery, while others require in vitro methods following isolation of the protein from the host cell.

It is understood that the proteins of the invention can be mixed with a pharmaceutically acceptable carrier, or diluted by a carrier, and/or enclosed within a carrier, which can, for example, be in the form of a capsule, sachet, paper or other container. When the carrier serves as a diluent, it can be a solid, semi-solid or liquid material which acts as a vehicle, excipient or medium for the active ingredient. Suitable pharmaceutically acceptable carriers include, for example, one or more of water, saline, phosphate buffered saline, dextrose, glycerol, ethanol and the like, as well as combinations thereof. Pharmaceutically acceptable carriers can further comprise minor amounts of auxiliary substances such as wetting or emulsifying agents, preservatives or buffers, which enhance the shelf life or effectiveness of the proteins. The compositions can, as is well known in the art, be formulated so as to provide quick, sustained or delayed release of the active ingredient.

The proteins of this invention can be in a variety of forms. These include, for example, solid, semi-solid and liquid dosage forms, such as tablets, pills, powders, liquid solutions, dispersions or suspensions, liposomes, suppositories, injectable and infusible solutions. Thus, the composition can be in the form of tablets, lozenges, sachets, cachets, elixirs, suspensions, aerosols (as a solid or in a liquid medium) or ointments containing for example up to 10% by weight of the active compound, soft and hard gelatin capsules, suppositories, injection solutions, suspensions, sterile packaged powders and as a topical patch. The preferred form depends on the intended mode of administration and therapeutic application. For parenteral administration by injection or infusion, a typical formulation will be in the form of a solution or suspension, possibly based on a lyophilized product.

Following purification, the presence and/or amounts or relative distribution of all the distinct members in the polyclonal antibody is typically assessed, for example by ion-exchange chromatography or using mass spectroscopy based methods, such as the marker peptide method described in WO 2006/007853. Methods for characterization of a polyclonal antibody composition are described in detail in WO 2006/007853. Determination of the presence and/or amount of each member of the polyclonal protein may also be performed at other points during the upstream or downstream manufacturing as desired. In one embodiment the method of the invention thus comprises at least one step during manufacturing and/or following purification to verify the presence and/or amount of each member of the polyclonal protein.

A further preferred feature of the distinct members of the polyclonal protein is protein homogeneity, so that the proteins can be purified easily. An ion exchange chromatography profile with one distinct peak is preferred for ease of characterisation. This applies to both the distinct members of the polyclonal protein and to the final drug product and drug substance composition. It is also preferable when combining the distinct members that they can be distinguished using ion exchange chromatography or other protein characterisation methods, so that the composition with all the distinct members can be characterised in one run. Thus, the distinct protein members are preferably chosen so as to allow identification of the distinct protein members in a characterisation step following purification.

The final drug product comprises the polyclonal protein formulated for administration (or possibly in lyophilized form suitable for reconstitution with e.g. sterile water prior to administration), a suitable packaging, and a package insert with prescription information as well as reference to a marketing authorization.

Expressing Proteins of Interest in Separate Organisms

By using this scenario, the advantages of using low cost upstream processing methods that rely on expression in vivo in an intact organism can be applied to the manufacture of polyclonal protein products. Recombinant expression of protein products in plants and animals is still in its infancy. Methods for expressing different members of a polyclonal protein product in one organism may prove to be difficult as several genes need to be inserted independently of each other in a cell that can subsequently divide and differentiate into an intact organism. It may therefore be advantage to be able to express only one distinct member of a polyclonal protein in one organism.

For animals, methods have been developed for inserting a heterologous expression construct into stem cells or egg cells and ensuring that the encoded product is subsequently expressed and secreted to the milk (in the case of mammals) or the egg (in the case of birds). The present invention can be extended to animals so that one animal expresses one member of the polyclonal protein, after which milk or eggs containing the protein product can be combined and subjected to a single downstream processing procedure. Alternatively, one or more steps of the downstream processing can be performed separately for one or more of the distinct protein members. The same species of animal will normally be used for expressing all members of the polyclonal protein in order to ease the downstream processing and subsequent characterisation.

For plants, methods for expression of heterologous proteins have likewise been developed. Some methods rely on expression of the product in a particular organ of the plant such as in a potato tuber, in the leaves, in the seeds, or in the flowers. Such organ-specific expression may ease the downstream processing, in particular if the fiber-rich parts of the plant can be avoided. Using such methods, recombinant protein products can be expressed in high quantities using low-cost methods for growing plants. A single plant will thus express one member of the polyclonal protein, and the different members may be combined prior to, during or after downstream processing. The same species and variety of plant will normally be used for expressing all members of the polyclonal protein.

Therapeutic Uses of Compositions Prepared According to the Invention

The pharmaceutical compositions prepared according to the present invention may be used for the treatment, amelioration or prevention of a disease in a mammal. Diseases that can be treated with the present pharmaceutical compositions include cancer, infectious diseases, inflammatory diseases, allergy, asthma and other respiratory diseases, autoimmune diseases, cardiovascular diseases, diseases of the central nervous system, metabolic and endocrine diseases, and transplantation rejections.

In addition, polyclonal antibodies produced in accordance with the invention may be used for diagnostic purposes, e.g. in diagnostic kits, and in kits for environmental use, e.g. for the detection of contaminants.

### EXAMPLES

### Example 1

### Separate Cell Expansion, Mixed Production in Shaker Flasks

Overview

Six different populations of cells were counted and mixed in equal cell ratios (1:1:1:1:1:1) and used to seed a bioreactor wherein each cell population produced a different antibody. The cells were cultivated for 7 days, after which the amount of each of the six antibodies was quantified using ion exchange chromatography (IEX). The experiment was repeated twice and the batch-to-batch reproducibility of the obtained polyclonal antibody composition was investigated.

Materials and Methods

Cell Line

The parental producer cell line used is a derivative of the DHFR-negative CHO cell line DG44 obtained from Lawrence Chasin, Columbia University. DG44 cells were transfected with a cDNA for the adenovirus type 5 transactivator E1A in the vector pcDNA3.1+ (Invitrogen). Transfectants were selected with Geneticin (Invitrogen) at a concentration of 500 μg/ml. After selection the cells were single-cell cloned by limiting dilution. Clones were tested for antibody expression by transient transfection with an antibody plasmid. A single clone showed an expression level in the transient assay that was improved by a factor of 3 compared to the untransfected DG44 cell line. In comparisons performed with stable transfection, selected pools showed a 4-5 times increased expression level compared to the wild-type DG44 cell line. This clone (termed ECHO) was sub-cloned twice and appeared to be stable with regard to antibody expression.

Antibody Expression Plasmids

The IgG1 antibody expression plasmids used were constructed so the coding regions for heavy (VH+gamma 1 constant region) and light chain (kappa 02-286) were expressed using two identical head-to-head human CMV promoters with a spacer element in between. Selection for transfectants was carried out using a mouse dihydrofolate reductase cDNA (DHFR) cassette driven by an internal ribosome entry site (IRES) located downstream the heavy-chain coding sequence. Six different antibodies were chosen that were directed against different Vaccinia virus surface proteins. Table 2 shows the antibodies and the ECHO clones expressing them.

TABLE 2
Antibodies and ECHO clones

| Antibody | ECHO clone name |
|---|---|
| Sym002-037 | 36-8 |
| Sym002-186 | 37-11 |
| Sym002-482 | 39-3 |
| Sym002-303 | 26-9 |
| Sym002-286 | 28-6 |
| Sym002-235 | 38-8 |

Transfection of ECHO Cells

ECHO cells were seeded in T80 flasks at a density of 0.3×10⁶ cells/per flask in MEM alpha medium (with nucleosides) (Invitrogen) with 10% fetal calf serum (FCS) (Invitrogen). Within an hour of seeding the cells were transfected with Fugene®6 (Roche):
10 μl of Fugene®6 was mixed with 490 μl Dulbecco's modified Eagle's medium and allowed to incubate for 5 min. at room temperature
5 μg of expression plasmid was added and the mix was incubated for a further 15 min. at room temperature
The mix was added to the cell culture flask

On the following day the medium with transfection reagents was aspirated, each flask was washed once with 5 ml of MEM alpha medium (without nucleosides) with 10% dialyzed FCS (Invitrogen) (MEMalpha-) and 10 ml of the same medium was added together with methotrexate at a concentration of 2 nM. The medium was changed twice a week. After 15 days the cells were trypsinized and all cells were transferred to new flasks.

For the production of single-cell clones the cells in the pools were stained for surface-associated antibody and FACS single-cell sorted using a FACS-Aria (Becton-Dickinson). After approximately 1 week wells were inspected by microscope for the presence of single clones. After approximately 2 weeks supernatants from wells with a single clone were assayed each in a single dilution by IgG ELISA and based on the ELISA value and visual inspection of the wells 24 clones representing each antibody were selected for adaptation to serum-free suspension culture.

Adaptation to Serum-Free Suspension Culture

Cells were trypsinized and counted. 5.0×10⁶ cells were centrifuged and resuspended in 10 ml ProCHO4™ serum-free medium (Lonza)+4 mM L-glutamine (Invitrogen)+ 1/100 MEM NEAA (Invitrogen). The cells were transferred to 50 ml cell culture tubes (TRP, Switzerland) and incubated on a shaker at 37° C. Cell densities were counted twice a week and each time the cultures were diluted to 0.5×10⁶ cells per ml (for the first 2 weeks) or to 0.3×10⁶ cells per ml (for the remaining period). After 4-5 weeks, the doubling time for most clones approached 30 hours, at which time point it was considered that they were adapted to serum-free culture. At the end of the adaptation period the cells were assayed by ELISA, frozen and used for expression experiments.

Thaw and Expansion

Each of the six different cells lines was thawed in ProCHO4™ medium heated to 37° C. The cell suspension was centrifuged 4 minutes at 800 rpm (160 G), the supernatant was removed, and the cell pellet was resuspended in 12 ml ProCHO4™ medium at 37° C. The cell suspension was then transferred to a T75 flask and placed in an incubator at 37° C. and 5% CO₂. On day 2 the culture was adjusted to 5.0×10⁵ cells/ml in ProCHO4™ medium in a shaker or a 50 ml vial/bioreactor. During cell expansion the cell concentration was adjusted to 0.5×10⁶ cells/ml twice or three times a week. After a week of expansion, each of the six cell lines was set up with 0.5×10⁶ cells/ml in 60-80 ml medium in 500 ml shakers.

Cultivation

When seeding the shakers, the six cell lines were mixed and the inoculum was divided into two 250 ml shakers. Briefly, before mixing, samples from each cell line were counted three times and the average viable cell count was used. A volume of each clone corresponding to 15.0×10⁶ cells was taken and the six volumes were mixed thoroughly and a new cell count was made. Based on this cell count, two 250 ml shakers with a (mixed) cell concentration of 0.5×10⁶/ml were set up. The calculated volume for each shaker was transferred to a 50 ml tube and centrifuged at 800 rpm (160 G) for 4 min. The supernatant was discarded, and the cells were resuspended in 50 ml ProCHO4™ medium and transferred to a 250 ml shaker. The shakers are then placed in an incubator at 37° C. and 5% CO₂ on a shaking table at 100 rpm and an amplitude of 2.6 cm.

The experiment was carried out on two separate days; day 1 with experiment numbers 1A and 1B and day 2 with experiment numbers 2A and 2B. The cultivation was run for 7 days, after which supernatants were saved for IEX profiling.

Analysis of IgG Composition

5-10 ml 0.22 μm filtered medium supernatant was loaded onto a 1 ml MabSelect SuRe™ column (GE Healthcare). The column was washed with 10 ml PBS pH 7.4 and eluted with 0.1 M glycine pH 2.7 as described by the manufacturer. Pooled protein material was dialyzed twice against 40 mM NaCl, 50 mM Na-acetate pH 5.0 and total IgG concentration was determined by measuring the absorbance at 280 nm.

60 μg IgG mixture was loaded onto the weak cation exchange column PolyCat A (100×4, 6 mm, 3 μm, 1500 Å) from PolyLC. The protein was eluted by applying a gradient from 150 to 500 mM NaCl in a Na-Acetate pH 5.0 buffer at a flow rate of 1 ml/min over 72 minutes. The 215 nm absorbance of the eluate was monitored and relative amounts of individual IgGs were determined by integration of the signal. In order to compare different samples, the total amount of antibodies was set to 100% and the ratio of each of the six antibodies was calculated.

Results

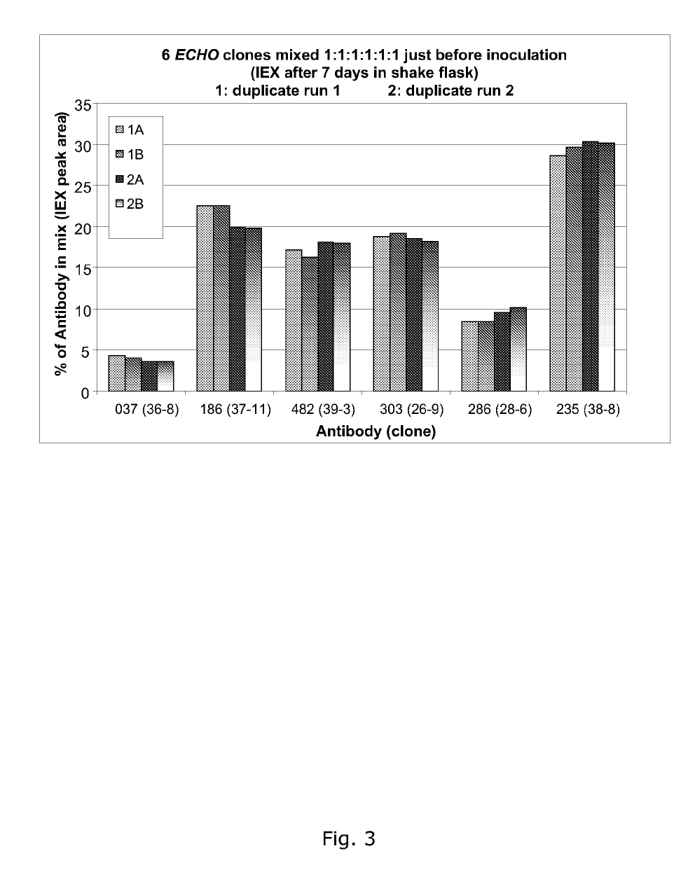

The supernatant from shaker flasks containing the mixed populations was harvested after 7 days and the IEX profiles were analyzed. The relative area of each antibody is given in Table 3 and FIG. 3.

TABLE 3
Relative area of each antibody after 7 days
of cultivation in a mixed population.

| | Relative areas (% of total amount of antibodies) | | | |
|---|---|---|---|---|
| Antibody | Mix experiment 1 | | Mix experiment 2 | |
| (clone no. in parenthesis) | 1A | 1B | 2A | 2B |
| 037 (36-8) | 4.3 | 4.0 | 3.6 | 3.6 |
| 186 (37-11) | 22.5 | 22.5 | 19.9 | 19.8 |
| 482 (39-3) | 17.2 | 16.3 | 18.0 | 18.0 |
| 303 (26-9) | 18.8 | 19.1 | 18.5 | 18.2 |
| 286 (28-6) | 8.5 | 8.5 | 9.6 | 10.2 |
| 235 (38-8) | 28.7 | 29.6 | 30.3 | 30.2 |
| Sum | 100.0 | 100.0 | 99.9 | 100.0 |

The table shows that
The variation in amounts of each of the six antibodies between two different shaker flasks mixed at the same time is very small. It is likely within the standard deviation limits of the method.
Cells mixed on different days also have a very small variation, indicating that it is possible to mix six different populations of cells in equal cell ratios and culture them together, resulting in the same relative amount of each of the six antibodies after cultivation.

### Example 2

### Separate Upstream Cultivation in Fed Batch Bioreactors, Mixing Before Purification

Overview

Five different ECHO cell lines expressing five different IgG antibodies were cultured individually for 13-14 days in bioreactors. After cultivation, antibody-containing fractions were mixed (2:1:1:1:1) during purification in order to obtain a composition of antibodies in the bulk drug substance (BDS).

Expression plasmids, cells etc. were as in Example 1 with the exception that only five antibodies were expressed (see Table 4).

TABLE 4
Antibodies and ECHO clones

| Antibody | ECHO clone name |
|---|---|
| Sym002-186 | 37-11 |
| Sym002-482 | 39-3 |
| Sym002-303 | 26-9 |
| Sym002-286 | 28-6 |
| Sym002-235 | 38-8 |

Thaw and Expansion

Each of the five different cell lines was thawed in ProCHO4™ medium heated to 37° C. The cell suspension was centrifuged 4 minutes at 800 rpm (160 G), the supernatant was removed, and the cell pellet was resuspended in 12 ml 37° C. ProCHO4™ medium. The cell suspension was then transferred to a T75 flask and placed in an incubator at 37° C. and 5% CO₂. On day 2 the culture was adjusted to 5.0×10⁵ cells/ml in ProCHO4™ medium in a shaker or a 50 ml tube “bioreactor”. During cell expansion the cell concentration was adjusted to 0.5×10⁶/ml twice or three times a week. After a week of expansion, each of the five cell lines was transferred to the serum-free production medium EX-CELL@ 302 (SAFC)+4 mM L-glutamine. The cell lines were set up with 0.5×10⁶ cells /ml in 60-80 ml medium in 500 ml shakers and cultivated for 2 weeks until bioreactor inoculation.

Cultivation and Mixing During Purification

Cultivations were carried out in six 500 ml working volume bioreactors (DASGIP AG) with automatic control of pH, dissolved oxygen, temperature, feeding profile and gas mixing. Each of the six bioreactors was inoculated with 5.0×10⁵/ml viable cells in EX-CELL® 302+4 mM L-glutamine. During the bioreactor runs, cells were fed on a daily basis with EX-CELL® 302 medium supplemented with a concentrated feed solution, glutamine and glucose to a final volume of 500 ml. The cultures were harvested after 13-14 days and after a clarification step (centrifugation at 1942 G for 15 min) each of the six supernatants was sterile filtrated through a 0.22 μm GP Express Plus Membrane Filter (Millipore).

Equal volumes of each of the filtrated supernatants were mixed in order to obtain a composition of antibodies. The antibody composition was purified by Protein A capture and analyzed by ion exchange chromatography (IEX).

Analysis of IgG Composition by Ion Exchange Chromatography

5-10 ml 0.22 μm filtered antibody composition was affinity purified by loading it onto a 1 ml MabSelect SuRe™ column (GE Healthcare). The column was washed with 10 ml PBS pH 7.4 and eluted with 0.1 M glycine pH 2.7 as described by the manufacturer. The purification was conducted on an Äkta Express system (GE Healthcare). Pooled protein material was dialyzed twice against 40 mM NaCl, 50 mM Na-acetate pH 5.0 and total IgG concentration was determined by measuring the absorbance at 280 nm.

80 μg IgG mixture was loaded onto a weak cation exchange column (PolyCat A, 100×4, 6 mm, 3 μm, 1500 Å) from PolyLC. The protein was eluted by applying a gradient from 150 to 500 mM NaCl in a Na-Acetate pH 5.0 buffer at a flow rate of 1 ml/min over 72 minutes. The 215 nm absorbance of the eluate was monitored, and relative amounts of individual IgG's were determined by integration of the signal. The total amount of antibodies was set to 100% and the ratio of each of the five antibodies was calculated.

Analysis of IgG Composition by HPLC

IgG content in supernatant samples from bioreactors was analyzed using an RX Daytona analyzer (Randox) and an immunoturbidimetric method according to the manufacturer's instructions.

Results

Cultivations were carried out in six 500 ml working volume bioreactors. Each of the six bioreactors was inoculated with 5.0×10⁵/ml viable cells, and harvested after 13-14 days of cultivation. The bioreactor cultivation setup, antibody concentration at harvest and volume fraction in the mixing step is given in Table 5. Antibody concentrations at harvest differ due to the different properties of the clones. The two cultivations with the same clone, CHM020-3 and CHM020-4, contained very similar antibody concentrations at harvest, indicating process robustness.

TABLE 5
Cultivation setup, antibody content at harvest
and volume fraction in the mixing step.

| | | | Antibody | | Antibody |
|---|---|---|---|---|---|
| | | | concentration | Volume | concentration |
| | | Expressed | at harvest | fraction | in mixed |
| Bioreactor | Antibody | in clone | (relative) | in mix | fraction (%) |
| CHM020-1 | Sym002-303 | 26-9 | 100 | ⅙ | 15.4 |
| CHM020-2 | Sym002-286 | 28-6 | 140 | ⅙ | 21.6 |
| CHM020-3 | Sym002-186 | 37-11 | 152 | ⅙ | 46.7 |
| CHM020-4 | Sym002-186 | 37-11 | 151 | ⅙ | |
| CHM020-5 | Sym002-235 | 38-8 | 35 | ⅙ | 5.4 |
| CHM020-6 | Sym002-482 | 39-3 | 71 | ⅙ | 10.9 |

The antibodies were mixed after clarification and sterile filtration by mixing equal volumes of all six cultivations. The antibody composition was then further purified by affinity chromatography and analyzed using cation exchange chromatography (CIEX). The relative amount of each of the five antibodies is given in Table 6 as estimated by CIEX chromatography.

TABLE 6
Relative amount of each antibody after
mixing as quantified by CIEX.

| | CIEX quantification |
|---|---|
| Antibody | of IgG in mix (%) |
| Sym002-303 | 17.4 |
| Sym002-286 | 23.9 |
| Sym002-186 | 47.9 |
| Sym002-235 | 3.8 |
| Sym002-482 | 7.0 |

The table shows that
Several antibodies can be mixed into a polyclonal antibody composition and purified using the described procedure.
A polyclonal protein made using this procedure can be adjusted to a predetermined composition by adjusting the amount of each antibody-containing fraction.

### Example 3

### Separate Seed Trains Followed by Single-Batch Fed Batch Production in Bioreactors

Overview

Six different populations of cells were counted and mixed in equal cell ratios (1:1:1:1:1:1) and used to seed a bioreactor, wherein each cell population produced a different antibody. The cells were cultivated for 12 days, after which the amount of each of the six antibodies in the bioreactor was quantified using ion exchange chromatography (IEX). The experiment was repeated 8 times and the variation among the mixed cell cultures was investigated.

Materials and Methods

Cell Line

The cell line used was the ECHO cell line described in Example 1.

Antibody Expression Plasmids

The IgG1 antibody expression plasmids used were constructed so the coding regions for heavy (VH+gamma 1 constant region) and light chain were expressed using two identical head-to-head human CMV promoters with a spacer element in between. Selection for transfectants was carried out using a mouse dihydrofolate reductase cDNA (DHFR) cassette driven by an internal ribosome entry site (IRES) located downstream of the heavy chain coding sequence. For the experiment six different antibodies (numbered as 810, 816, 824, 853, 856 and 894) that were directed against respiratory syncytial (RS) virus antigens F and G were chosen.

Transfection of ECHO Cells

The transfection method follows the transfection method described in Example 1.

Adaptation to Serum-Free Suspension Culture

Cells were trypsinized and counted. 5.0×10⁶ cells were centrifuged and resuspended in 10 ml ProCHO4™ serum-free medium (Lonza)+4 mM L-glutamine (Invitrogen)+ 1/100 MEM NEAA (Invitrogen). The cells were transferred to 50 ml cell culture tubes (TPP, Switzerland) and incubated on a shaker at 37° C. Cell densities were counted twice a week, and each time the cultures were diluted to 0.5×10⁶ cells per ml (for the first 2 weeks) or to 0.3×10⁶ cells per ml (for the remaining period). After 4-5 weeks the doubling time for most clones approached 30 hours, at which point it was considered that they were adapted to serum-free culture. The cells were then transferred to PowerCHO2™ serum-free medium (Lonza)+4 mM L-glutamine (Invitrogen)+ 1/100 MEM NEAA (Invitrogen). After 2-3 weeks, when the doubling time was below 35 hours, the cells were considered adapted, after which they were assayed for antibody productivity by ELISA, and frozen and used for expression experiments.

Thaw and Expansion

Each of the six different clones was thawed in PowerCHO2™ medium heated to 37° C. The cell suspension was centrifuged 4 minutes at 800 rpm (160 g), the supernatant was removed, and the cell pellet was resuspended in 12 ml 37° C. PowerCHO2™ medium. The cell suspension was then transferred to a T75 flask and placed in an incubator at 37° C. and 5% CO₂. On day 2 the culture was adjusted to 5.0×10⁵/ml in PowerCHO2™ medium in a shaker or a 50 ml cell culture tube. During cell expansion the cell concentration was adjusted to 0.5×10⁶/ml twice or three times a week. After 31 days of expansion, each of the six clones was set up in two different shaker flasks (series A and B), giving a total of 12 shaker flasks, with 0.5×10⁶/ml in 60-80 ml in 500 ml shakers.

Cultivation

After a total of 35 days of seed train expansion of the individual antibody producer cell lines, each of 4 bioreactors were seeded with a mixture of the six clones. This experiment was repeated twice, giving a total of 8 bioreactors as described below. Briefly, before mixing, each clone was counted three times and the average viable cell count was used. A volume of each clone corresponding to 25.0×10⁶ cells was taken and the ‘six clone mix’ was mixed thoroughly and a new cell count was made. Based on this cell count, a 500 ml bioreactor with a (mixed) cell concentration of 0.3×10⁶/ml was set up by adding PowerCHO2™ medium+20% (w/w) of a feed medium. The rest of the ‘six clone mix’ was sampled for IEX profile analysis. The bioreactor was then cultivated with controlled DO at 30%, pH 7.0±0.1, 80 rpm. Samples were taken out for IEX profiling after 4, 8 and 12 days.

The Eight Bioreactors

In order to investigate the robustness and batch-to-batch consistency of the process, clones from the 12 shaker flasks were used to seed 8 bioreactors as follows:

For bioreactor 1, cells were taken from each of the six clones of shaker flask seed train series A.

For bioreactor 2, cells were taken independently from shaker flask seed train series A in the same manner as for bioreactor 1.

For bioreactor 3, cells were taken from each of the six clones of shaker flask seed train series B.

For bioreactor 4, cells were taken independently from shaker flask seed train series B in the same manner as for bioreactor 1.

For bioreactors 5 to 8, cells were taken independently in the same manner as described for bioreactors 1 to 4. Bioreactors 5 to 8 were seeded two days after bioreactors 1 to 4.

Antibody Purification and CIEX Analysis of IgG Composition

5-10 ml 0.22 μm filtered medium supernatant was loaded onto a 1 ml MabSelect SuRe™ column (GE Healthcare). The column was washed with 10 ml PBS pH 7.4 and eluted with 0.1 M glycine pH 2.7 as described by the manufacturer. Pooled protein material was dialyzed twice against 40 mM NaCl, 50 mM Na-acetate pH 5.0, and total IgG concentration was determined by measuring the absorbance at 280 nm.

60 μg IgG mixture was loaded onto the weak cation exchange column PolyCat A (100×4, 6 mm, 3 μm, 1500 Å) from PolyLC. The protein was eluted by applying a gradient from 150 to 500 mM NaCl in a Na-Acetate pH 5.0 buffer at a flow of 1 ml/min over 72 minutes. The 215 nm absorbance of the eluate was monitored and relative amounts of individual IgGs were determined by integration of the signal. In order to compare different samples, the total amount of antibodies was set to 100% and the relative amount of each of the six antibodies was calculated.

Results

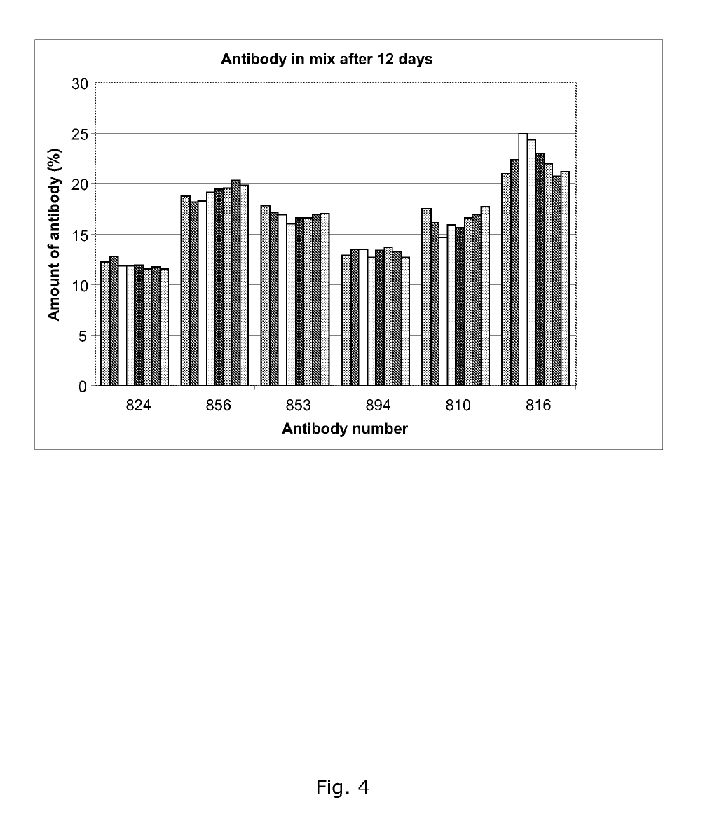

The supernatants from the 8 bioreactors containing the mixed populations were harvested after 12 days and the IEX profiles were analyzed after filtration and protein A capture purification. The relative area of each antibody is given in Table 7 and FIG. 4.

TABLE 7
Relative area of each antibody after 12 days
of cultivation in a mixed population.

| | Relative area (% of total | Standard deviation |
|---|---|---|
| Antibody | amount of antibodies) | as % of area |
| 810 | 16.4 | 6.1% |
| 816 | 22.4 | 6.9% |
| 824 | 11.9 | 3.4% |
| 853 | 16.9 | 2.9% |
| 856 | 19.2 | 4.0% |
| 894 | 13.2 | 3.0% |

Table 7 shows that the variation in the amounts of each of the six antibodies produced in a mixed culture is very small (presumably within detection limits of the method), in spite of the fact that the mixed cultures were seeded with clones from different shaker flasks, on different days, and with cells taken out independently of each other.

The IEX chromatograms in FIG. 4 show the relative amount of each antibody after 12 days of cultivation. Despite the deliberate variation in setting up the bioreactors, the resulting distributions for each of the 6 antibodies in the 8 bioreactors are highly similar, indicating that the process is robust and highly repeatable. In addition, this example demonstrates that the 8 separate cultivations could be carried out without any technical problems, suggesting that the process is suitable for large-scale manufacturing of a polyclonal antibody or other polyclonal protein.

### Example 4

### Separate Production in Animals, Mixing Before DSP

IgG heavy and light chain genes are cloned into an appropriate expression vector depending on the animal of choice, with expression of the two genes being under the control of beta casein promoters that direct gene expression to the mammary gland, and using a selectable marker gene. The expression construct is transfected (e.g. using LipofectAMINE™, or similar) into an appropriate donor cell line (e.g. fetal fibroblast cells). Selection marker resistant cells are selected by subsequent culture in appropriate medium. Individual cell lines are assayed by PCR and Southern blotting to detect the presence of both transgenes. Further characterization of candidate cell lines by fluorescence in situ hybridization (FISH) should confirm co-localization of the heavy and light chain transgenes on a single chromosome. The correctly transfected cell lines are used in somatic cell nuclear transfer (SCNT), using electrofusion to enucleated oocytes (cytoplasts). SCNT blastocysts are then transferred into the uterus with corpus luteum from the recipient animal. Recipients are returned to the herd to await subsequent evaluations for pregnancy determination. The offspring are analyzed by PCR analysis of skin biopsies for the IgG H and L genes.

The positive cloned offspring are subjected to a hormonal lactation and are milked to collect samples to assay for IgG expression. Cloned animals with satisfying IgG levels in the milk are used as founder animals for the production herd. A herd for each antibody is generated using this method. The milk from this collection of herds is analysed for IgG levels by ELISA (or similar) and is stored at approx. 4° C. in hold tanks. Mixing of the milk prior to purification may be done based on the IgG levels, or alternatively the milk batches can be purified separately.

Similar methods can be applied to manufacture in intact plants.

1. A method for manufacturing a drug product comprising at least two distinct protein members of a polyclonal protein, said method comprising the steps of:
a) providing at least two populations of cells, wherein each population encodes one distinct member of the polyclonal protein and is enclosed in a physically separate container comprising culture medium and cells expressing the protein, wherein the method comprises an upstream part comprising the steps of:
i) expanding the at least two populations of cells in one or more steps of a seed train in separate containers;
ii) expanding cells from the seed train in one or more steps of an inoculum train;
iii) culturing cells from the inoculum train in a production phase under conditions favoring expression of the protein members so as to express the at least two distinct protein members;
b) harvesting the expressed protein;
c) performing at least one downstream purification step on the harvested protein;
d) obtaining purified drug substance; and
e) formulating purified drug substance into a polyclonal drug product
wherein the at least two populations of cells are kept separate at least during the seed train, and wherein at least part of the downstream purification is carried out on a mixture comprising the individual members of the polyclonal protein.

2. The method of claim 1, wherein the at least two populations of cells are kept separate at least up to and including the inoculum train.

3. The method of claim 1, wherein the at least two populations of cells are kept separate at least up to and including the production phase.

4. The method of claim 1, wherein the at least two distinct protein members expressed by the at least two populations of cells are kept separate at least up to and including the protein harvest.

5. The method of claim 1, wherein the at least two distinct protein members expressed by the at least two populations of cells are kept separate at least up to and including at least one purification step.

6. (canceled)

7. The method of claim 1, wherein equal numbers of cells of the at least two populations of cells are combined.

8. The method of claim 1, wherein different numbers of cells of the at least two populations of cells are combined.

9. The method of claim 8, wherein cells from different populations are combined to obtain a polyclonal cell population capable of expressing approximately equal amounts of the distinct members of the polyclonal protein.

10. The method of claim 1, wherein cells from the at least two populations of cells are mixed in a pre-defined ratio.

11. The method of claim 10, wherein cells from the at least two populations of cells are combined in a ratio giving a pre-defined ratio of the distinct protein members in the drug substance or product.

12. The method of claim 1, wherein equal volumes from cell cultures of the distinct members of the polyclonal protein are combined.

13. The method of claim 1, wherein equal quantities of the distinct members of the polyclonal protein are combined.

14. The method of claim 1, wherein the at least two distinct members of the polyclonal protein are combined in a ratio giving a pre-defined ratio of the distinct protein members in the drug substance or drug product.

15. The method of claim 1, wherein at least one distinct protein member is kept separate from the other member(s) during at least one initial purification step.

16. The method of claim 1, wherein the separate part of the manufacturing method is performed in parallel for at least two distinct populations of cells and/or at least two distinct protein members expressed by the at least two populations of cells.

17. The method of claim 1, wherein the separate part of the manufacturing method is performed serially for at least two distinct populations of cells and/or at least two distinct protein members expressed by the at least two populations of cells.

18. (canceled)

19. The method of claim 1, wherein the separate part of the manufacturing method is performed using the same medium ingredients and process parameters for expression of different protein members.

20. The method of claim 1, wherein the at least two populations of cells are identical except for differences in expression vector sequences that encode the distinct protein members.

21. The method of claim 20, wherein the at least two populations of cells are identical except for differences in at least one expression vector sequence that encodes a variable region of the distinct protein members.

22. The method of claim 20, wherein at least one distinct member of the polyclonal protein comprises one constant region, and at least one other distinct member comprises a different constant region.

23. The method of claim 1, wherein at least one distinct member of the polyclonal protein comprises one glycosylation pattern, and at least one other distinct member comprises a different glycosylation pattern.

24. The method of claim 1, wherein the polyclonal protein comprises at least three distinct members

25. The method of claim 1, wherein the polyclonal protein is secreted.

26. The method of claim 25, wherein the polyclonal protein is a polyclonal antibody or polyclonal antibody fragment.

27. The method of claim 1, wherein the polyclonal protein is a multimeric protein.

28. The method of claim 1, wherein the cells are prokaryotic.

29. The method of claim 1, wherein the cells are eukaryotic.

30. The method of claim 29, wherein the eukaryotic cells are from a eukaryotic organism selected from the group consisting of plants, yeast, fungi, vertebrates and invertebrates.

31. The method of claim 29, wherein the eukaryotic cells are mammalian cells.

32. The method of claim 31, wherein the mammalian cells are selected from the group consisting of Chinese hamster ovary (CHO) cells, COS cells, BHK cells, myeloma cells, NIH 3T3 cells, fibroblast cells, amnion cells, immortalised human cells such as HeLa cells, HEK293 cells and PER.C6 cells.

33. The method of claim 1, further comprising at least one step during manufacturing and/or following purification to verify the presence and/or amount of each member of the polyclonal protein.
